(19) United States
(12) Patent Application Publication
(10) Pub. No.: US 2020/0245967 A1
(43) Pub. Date: Aug. 6, 2020

(54) LOCALIZATION OF BLEEDING

(71) Applicant: General Electric Company, Schenectady, NY (US)

(72) Inventors: Prem Venugopal, Clifton Park, NY (US); Luca Marinelli, Schenectady, NY (US)

(21) Appl. No.: 16/267,147

(22) Filed: Feb. 4, 2019

(51) Int. Cl. A61B 8/06; A61B 8/08; A61B 5/02

(52) U.S. Cl. CPC A61B 8/06; A61B 8/488; G01S 15/8981; A61B 8/0891; A61B 5/02007; A61B 5/02042

(57) ABSTRACT

The present disclosure relates to localization of bleeds (e.g., arterial bleed events) using a limited or minimal number of ultrasound scans. In one implementation, Doppler ultrasound is used to measure blood flow velocities in a one-dimensional (1D) arterial tree model to determine the location and size of bleed. In a second implementation, ultrasound measured waveforms for blood flow velocity and vessel cross-sectional area are de-composed. The features in the de-composed waveforms are then used to locate the bleed using a trained algorithm.

### BACKGROUND

The subject matter disclosed herein relates to localizing bleeding, both internal and external, including localizing such occurrences outside of a hospital environment.

Vascular trauma with vessel disruption can occur in a variety of environments, including both military and civilian environments. In some instances, the vascular trauma may be internal, without a clear break (e.g., an entry or exit wound) in the skin corresponding to the location of the trauma. In such circumstances, it may be difficult to localize where in the body an internal bleeding event is occurring so that treatment can be applied or, indeed, if there is internal bleeding occurring at all. Even in the presence of entry and exit wounds, it may be difficult to ascertain which blood vessel was affected and the location of the bleed.

For example, a skilled or trained person may be able to determine if a severe bleed event is present based on indications of vascular injury that include pulsatile hemorrhage, expanding hematoma, bruit or thrill over the injury site, absent extremity pulses, and arterial pressure index<0.9. However, such indications may be insufficient to make such a determination even by a trained individual, and likely would be impossible or impractical for an untrained individual to evaluate. Further, even to the extent these factors may allow a skilled or trained person to determine if a vascular injury is present, they may be still insufficient to localize the internal site of the vascular trauma, which is necessary to apply treatment.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible embodiments. Indeed, the invention may encompass a variety of forms that may be similar to or different from the embodiments set forth below.

In one embodiment, a method is provided for localizing a vascular bleed. In accordance with this embodiment, blood flow velocities are measured at one or more locations on a body of a patient. The blood flow velocities are fit to a vascular tree model. Based upon the fit of the blood flow velocities to the vascular tree model, one or both of a location or a size of a bleed event within the patient are determined.

In a further embodiment, another method is provided for localizing a vascular bleed. In accordance with this embodiment, one or more waveforms are generated using ultrasound imaging. The one or more waveforms describe one or both of vascular vessel cross-sectional area at one or more locations or blood flow velocity at the one or more locations. The one or more waveforms or components or features derived from the one or more waveforms are input to a trained machine learning algorithm. An output of the trained machine learning algorithm is one or both of a location or a size of a bleed event within a patient.

In another embodiment, a system for localizing bleed events is provided. In accordance with this embodiment, the system comprises an ultrasound scanner configured to generate ultrasound data at one or more locations of a body of a patient; a memory component configured to store one or more processor-executable routines; and a processing component configured to receive or access the ultrasound data and to execute the one or more processor-executable routines. The one or more routines, when executed by the processing component, cause the processing component to perform acts comprising: measuring at least blood flow velocities at one or more locations on a body of a patient; and processing at least the blood flow velocities or one or more components or features derived from at least the blood flow velocities to determine one or both of a location or a size of a bleed event within the patient.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is an embodiment of a block diagram of an ultrasound system, in accordance with aspects of the present disclosure;

FIG. 2 schematically represent aspects of a bleed event model, in accordance with aspects of the present disclosure;

FIG. 3 depicts a patient vasculature and representative ultrasound waveforms that might be observed for a bleed event, in accordance with aspects of the present disclosure;

FIG. 4 depicts a patient vasculature and representative ultrasound waveforms that might be observed for a bleed event, in accordance with aspects of the present disclosure;

FIG. 5 depicts a process flow for locating and/or estimating the size of a bleed event, in accordance with aspects of the present disclosure;

FIG. 6 depicts an example of a baseline scenario waveform decomposed into forward and backward components, in accordance with aspects of the present disclosure;

FIG. 7 depicts wave intensities for the baseline scenario, in accordance with aspects of the present disclosure;

FIG. 8 depicts an example of a bleed event scenario waveform decomposed into forward and backward components, in accordance with aspects of the present disclosure;

FIG. 9 depicts wave intensities for the bleed event scenario of FIG. 8, in accordance with aspects of the present disclosure;

FIG. 10 depicts another example of a bleed event scenario waveform decomposed into forward and backward components, in accordance with aspects of the present disclosure;

FIG. 11 depicts wave intensities for the bleed event scenario of FIG. 10, in accordance with aspects of the present disclosure;

FIG. 12 depicts a process flow illustrating steps of one example of a process for training a machine learning algorithm, in accordance with aspects of the present disclosure; and

FIG. 13 depicts a process flow illustrating steps of one example of a process for using a trained a machine learning algorithm to locate and/or estimate the size of a bleed event, in accordance with aspects of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

As discussed herein, bleeding, whether internal or external, can be difficult to diagnose and, if present, difficult to localize for appropriate treatment. Doppler ultrasound has been used in some circumstances to detect the presence of arterial injuries and can also be used for localization of the injury. However, the scanning process can be time consuming, especially if the bleed location (e.g., a puncture or tear in the blood vessel) is less than obvious, and may require a person trained in ultrasound to perform.

With this in mind, the present technique relates to localization of bleeds (e.g., blood vessel bleed events, such as punctures or tears in an artery) using fewer or a minimum number of ultrasound scans. In one implementation, doppler ultrasound measured blood flow velocities are used in a one-dimensional (1D) arterial tree model to determine the location and size of bleed. In a second implementation, ultrasound measured waveforms for blood flow velocity and vessel cross-sectional area are de-composed into forward and backward running wave components. The features in the de-composed waveforms are then used to locate the bleed using a trained machine learning algorithm.

Regardless of whether the first or the second technique is employed, once the bleed has been detected and accurately localized, a treatment plan can be formulated and therapy to contain blood loss delivered. Conventional therapeutic approaches at the point of care may include application of pressure or hemostatic pads, but additional therapies are being explored. For example, balloon catheters for bleeds in major arteries have recently been developed. Detailed information about the location of the bleed as may be acquired using the present techniques would enable deployment of such catheters in a location to maximize therapeutic effectiveness and to minimize side effects.

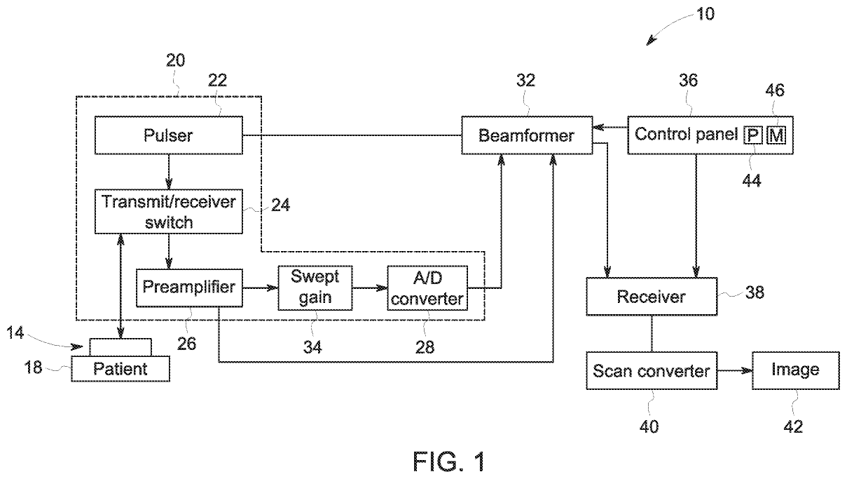

With the preceding in mind, and by way of providing useful context, FIG. 1 depicts a high-level view of components of an ultrasound system 10 that may be employed in accordance with the present approach. The illustrated ultrasound system 10 includes a transducer array 14 having transducer elements suitable for contact with a subject or patient 18 during an imaging procedure. The transducer array 14 may be configured as a two-way transducer capable of transmitting ultrasound waves into and receiving such energy from the subject or patient 18. In such an implementation, in the transmission mode the transducer array elements convert electrical energy into ultrasound waves and transmit it into the patient 18. In reception mode, the transducer array elements convert the ultrasound energy received from the patient 18 (backscattered waves) into electrical signals.

Each transducer element is associated with respective transducer circuitry, which may be provided as one or more application specific integrated circuits (ASICs) 20, which may be present in a probe or probe handle. That is, each transducer element in the array 14 is electrically connected to a respective pulser 22, transmit/receive switch 24, preamplifier 26, swept gain 34, and/or analog to digital (A/D) converter 28 provided as part of or on an ASIC 20. In other implementations, this arrangement may be simplified or otherwise changed. For example, components shown in the circuitry 20 may be provided upstream or downstream of the depicted arrangement, however, the basic functionality depicted will typically still be provided for each transducer element. In the depicted example, the referenced circuit functions are conceptualized as being implemented on a single ASIC 20 (denoted by dashed line), however it may be appreciated that some or all of these functions may be provided on the same or different integrated circuits.

Also depicted in FIG. 1, a variety of other imaging components are provided to enable image formation with the ultrasound system 10. Specifically, the depicted example of an ultrasound system 10 also includes a beam former 32, a control panel 36, a receiver 38, and a scan converter 40 that cooperate with the transducer circuitry to produce an image or series of images 42 that may be stored and/or displayed to an operator or otherwise processed as discussed herein. A processing component 44 (e.g., a microprocessor) and a memory 46 of the system 10, such as may be present control panel 36, may be used to execute stored routines for processing the acquired ultrasound signals to generate meaningful images and/or motion frames, which may be displayed on a monitor of the ultrasound system 10, and/or to localize and/or estimate the size of a bleed event, such as a severe bleed event, as discussed herein.

With the preceding system level discussion in mind as useful context, the present techniques may utilize such an ultrasound system to acquire raw data or reconstructed images that may be processed as discussed herein to localize the location of bleeding in a patient.

By way of example, in accordance with a first technique, vascular tree model is employed. While the vascular tree described in the examples below is an arterial tree, the methods and approaches described herein could also be applied to venous trees or a combined tree consisting of both arteries and veins. In one implementation the arterial tree model has multiple segments (e.g., 55 connected segments). Blood flow in the segments of the arterial tree model is modeled using a one-dimensional (1D) wave propagation model. Though 1D models are discussed herein to simplify explanation and illustration of the present concepts, the present technique may also be extended to use with two-, three-, or four-dimensional models (i.e., 2D, 3D, or 4D) and should therefore be understood to not be limited to the present 1D model examples. In one example, flow rate, pressure, and cross-sectional area are modeled or solved for by the arterial tree model as part of modeling blood flow. Terminal segments in the arterial tree model may have a zero-dimensional (0D) lumped model at the outlet modeling the terminal resistance and compliance. The heart may also be modeled using a 0D lumped model, such as a time-varying elastance model that may be used to simulate cardiac contraction.

Using this arterial tree model, 1D models of arterial bleeds may be simulated. By way of example, in studies performed in accordance with the present technique, bleeds of different sizes (2 mm in diameter, 2.5 mm in diameter and 4 mm in diameter) were modeled in the right femoral artery. In particular, the resistance to flow due to pressure drop across the bleed was modeled by treating the bleed as an orifice through which the blood expands into a much larger volume. This enables a larger size bleed to bleed more than a smaller size bleed. The pressure drop is modeled using the following equation:

$$Δ p = \frac{ρ {K}_{t}}{2 {A}_{0}^{2}} {( \frac{{A}_{0}}{{A}_{s}} - 1 )}^{2} \vert Q \vert Q .$$

Here Aₛ is the bleed area, A₀ is the larger area into which the blood is expanding, Q is the flow rate, ρ is the density of blood and Kₜ is a constant. Tissue resistance seen by the bleed was also modeled using an assumption that tissue is a porous media with permeability K. Under these assumptions, tissue resistance is given by the following equation:

$$R = \frac{μ L}{K A} .$$

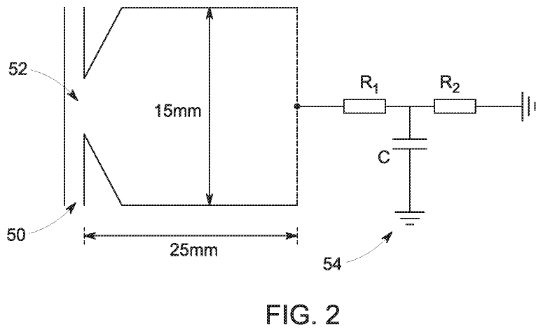

Here μ is the blood viscosity, L is the length of the porous media, A is its cross-sectional area and K is the permeability associated with the tissue. The cross-sectional area, A₀, in the pressure-drop equation is assumed to be the same as the porous media cross-sectional area, A. A conceptualized view of this model is shown in FIG. 2 in which artery 50 (e.g., the femoral artery) has a puncture or tear (e.g., bleed 52). In the example shown on FIG. 2, the porous media is assumed to have a length of 25 mm and cross-sectional area of 0.0007 mm². The 1D model is terminated with a 0D lumped model and the total lumped model resistance is set to the tissue resistance, i.e.

$${R}_{1} + {R}_{2} = \frac{μ L}{K A} .$$

The terminal pressure is assumed to be zero, though in reality it will increase as blood starts to pool in the tissue. The tissue resistance seen by the bleed is lower than the microcirculation resistance. The bleed thus offers a low resistance path to the blood flow causing blood to be diverted to this location (though this effect may be temporarily dampened in real world scenarios due to autoregulation mechanisms).

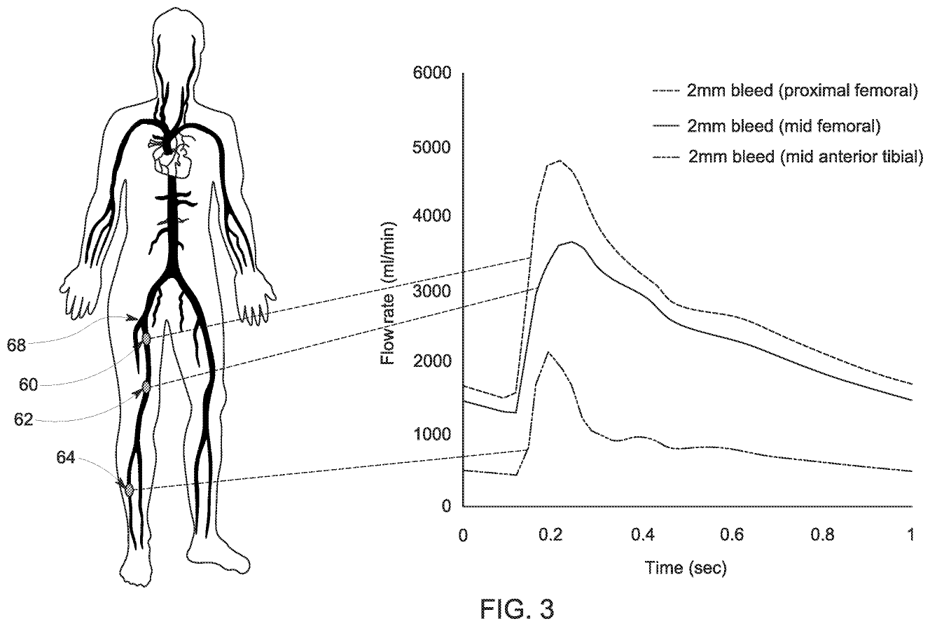

The effects of this diversion of blood in response to a bleed event may manifest by impacting or otherwise influencing the arterial waveform seen for that arterial segment or a connected arterial segment. In this manner, an arterial flow waveform can be used to locate the bleed within the arterial tree. By way of example, and turning to FIG. 3, in the depicted scenario three different scenarios are illustrated with the same size bleed (i.e., 2 mm) at three different locations along the right leg (i.e., proximal femoral 60, mid-femoral 62, and mid anterior tibial 64). Flow waveforms observed at the femoral-deep femoral bifurcation 68 is monitored and the waveform observed at this location for each bleed location is shown in FIG. 3. As may be observed, the waveform shape changes depending on the bleed location. In this case the shape changes as more blood bleeds out of the proximal bleed compared to the distal bleed, even though the bleed sizes are the same. This may be attributed to the head (i.e., total pressure) at the proximal site bleed being larger than the distal site.

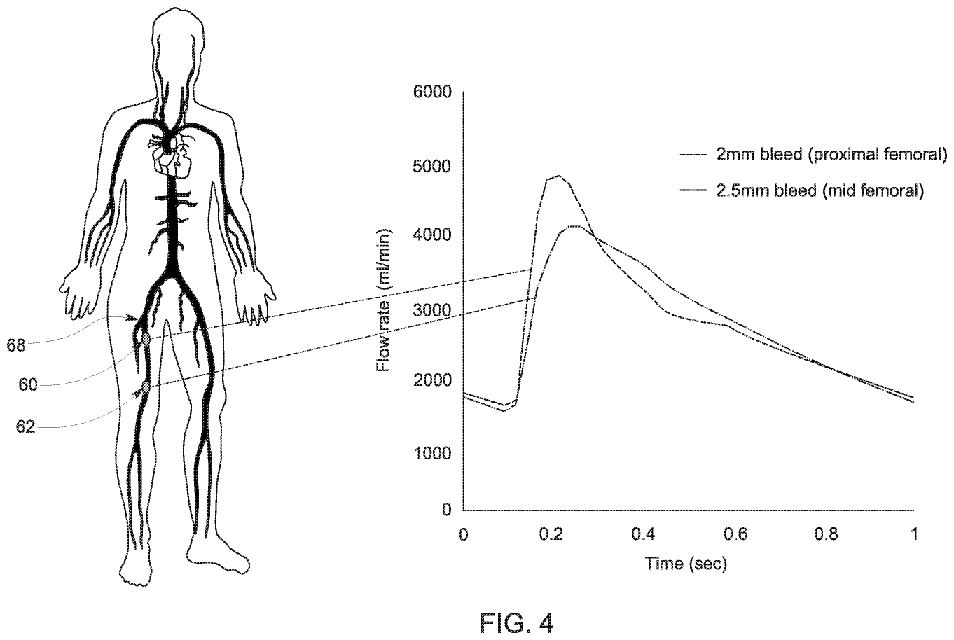

In the preceding example, the waveform shape changes because there is more bleed-out at the proximal (i.e., upstream) site compared to the distal (i.e., downstream) site. However, even when the amount of bleeding at the two sites are the same, the waveform shapes can be different. Consider FIG. 4, where bleed events are modeled in both the proximal femoral artery 60 and the mid-femoral artery 62. In this example, the bleed sizes are not the same. In particular, to have the bleed flow rate at the two locations be almost the same, the mid-femoral artery bleed is made slightly larger than the proximal femoral bleed (2.5 mm versus 2 mm). As in the preceding example, the flow waveforms are monitored at the femoral-deep femoral bifurcation 68 and still shows differences between the two cases, as shown in FIG. 4. This may be taken as an indication that the bleed location is encoded in the flow waveform and can be used to locate the bleed.

With the preceding in mind, and turning to FIG. 5 in accordance with aspects of a first technique discussed herein, a 1D arterial tree model 80 is constructed (step 82) based on average or expected human measurements for the vasculature in question (such as taking into account patient demographic factors such as age, gender, body mass index, and so forth) or, optionally, based on ultrasound measurements (step 84) of vessel cross sectional areas 86 acquired at one or more locations (e.g., at the one or more locations where blood flow velocities are measured using ultrasound).

Based on the 1D arterial tree model a bleed event (e.g., blood vessel puncture or tear) may be localized and its size determined by measuring (step 90) blood flow velocities 92, such as using Doppler ultrasound, at one or more locations If the bleed location is approximately known, the ultrasound measurements may be made upstream or downstream of the bleed. On the other hand, if it is suspected that there is a bleed in the legs for example and the bleed location is completely unknown, then Doppler measurements can be made at a few landmark locations. The landmark locations could include the left and right knees, the left and right ankle and the upper portion of the left and right thighs, close to the groin. In addition, to blood flow velocities 82, additional information may be optionally acquired. For example, one or both of blood pressure 96 or heart rate 98 may be measured (step 94) using suitable methodologies.

The measured blood flow velocities 92 and any additional acquired blood pressure or heart rate data may be fit (step 100) to the 1D arterial tree model to determine the size and location of the bleed event (block 102). By way of example, in one embodiment the location and size of the bleed in the 1D arterial tree model 80 may be optimized to minimize the error between predicted and measured blood flow velocities 92.

While the preceding relates to one technique for determining the location and size or severity of a bleed event, Additional embodiments are also contemplated. For example, a second technique for localization of a bleed event using waveforms may be based on the observation that the bleed event (e.g., puncture or tear in a blood vessel, such as an artery) generates pulse wave reflections that may be extracted from the original waveform and used to locate the bleed.

For example, given pressure (p) and velocity (u) waveforms that may be generated using an ultrasound modality, the forward and backward running contributions can be written as:

$$\begin{matrix}d {p}_{\pm } = \frac{1}{2} ( d p \pm ρ c ( p ) du ) & ( 2 ) \\ d {u}_{\pm } = \frac{1}{2} ( d u \pm \frac{d p}{ρ c ( p )} ) & ( 3 )\end{matrix}$$

Here the subscripts “+” and “−” denote the forward and backward running components (i.e., waves) respectively, c is the pulse wave velocity, and ρ is the blood density. While the above decomposition is written in terms of pressure and velocity, it could also be written in terms of area and velocity, both quantities that can be measured using ultrasound imaging.

Based on the above decomposition, one can define a quantity called wave intensity for the forward and backward running waves as:

$${W}_{\pm } = \frac{d {p}_{\pm }}{d t} \frac{d {u}_{\pm }}{d t} = \pm \frac{ρ c ( p )}{4} {( \frac{d R \pm }{d t} )}^{2} \qquad ( 4 )$$

In this context, the wave intensity is positive for forward running waves and negative for backward running waves.

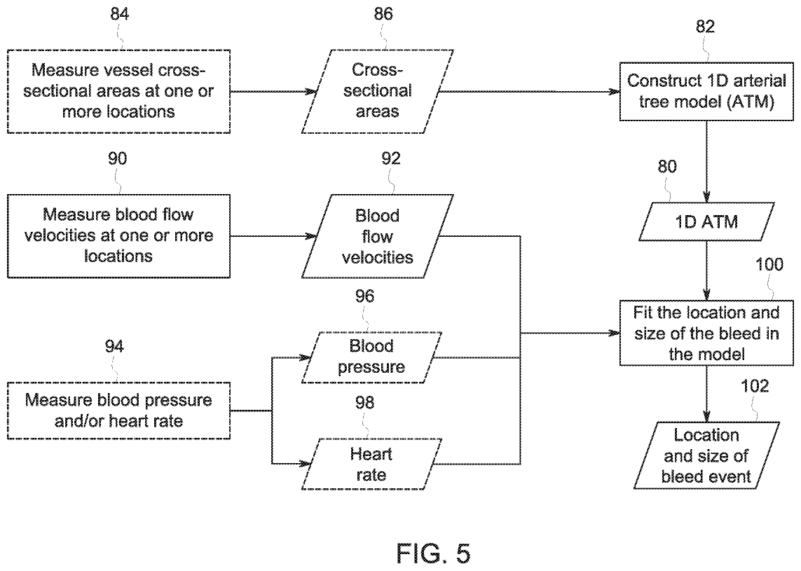

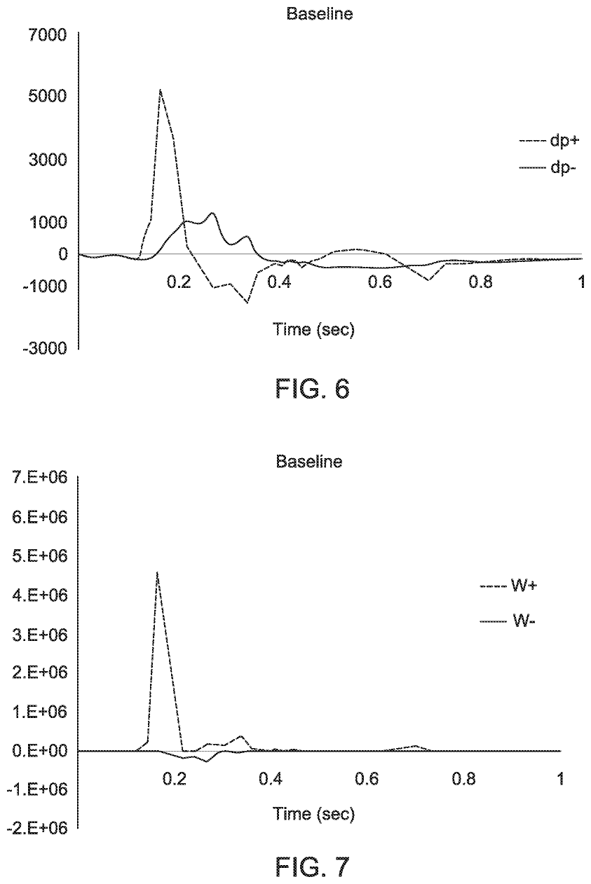

With the preceding in mind, FIGS. 6 and 7 depict the pressure waveforms obtained using a 1D arterial tree model decomposed into forward (+) and backward (−) components (FIG. 6) and the corresponding wave intensities (FIG. 7). The waveforms shown in FIGS. 6 and 7 correspond to a baseline scenario without a bleed event and obtained at the proximal femoral artery. Based on the signs of the decomposed pressure waveforms one can identify certain features in the forward and backward running wave intensity profiles, such as a forward compression wave corresponding to when the forward component of the decomposed pressure is positive (i.e., dp+>0), a forward de-compression wave when the forward component of the decomposed pressure is negative (i.e., dp+<0), and a backward compression wave when the backward component of the decomposed pressure is positive (i.e., dp− above 0).

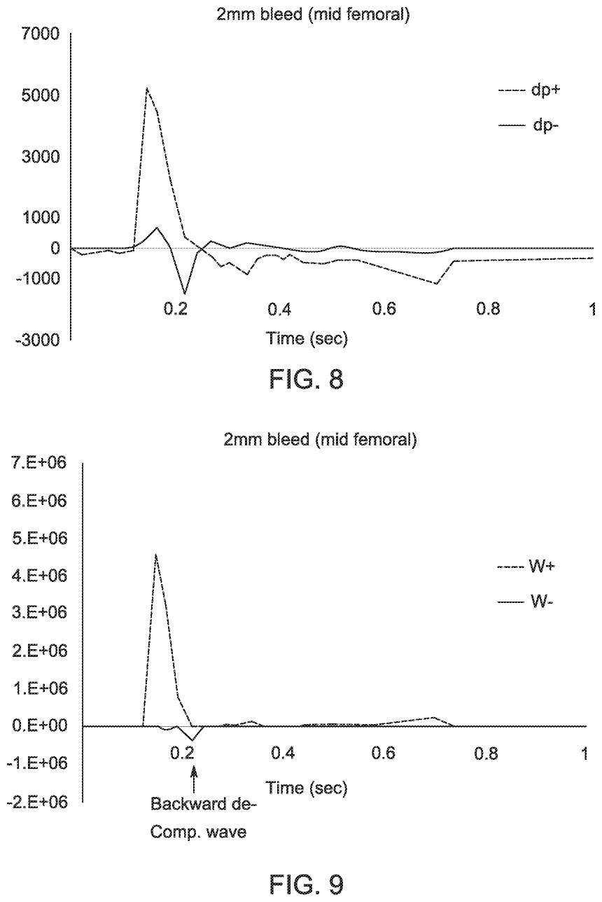

Conversely, a scenario involving a bleed is illustrated in FIGS. 8 and 9. In particular, in this scenario, a 2 mm bleed in the mid-femoral artery is modeled and waveforms obtained at the proximal femoral artery. As in the baseline scenario, the pressure waveforms (FIG. 8) and wave intensities (FIG. 9) obtained using the 1D model are decomposed into forward and backward components. FIGS. 8 and 9 depict these waveforms in respective alignment with their counterparts in FIGS. 6 and 7. As may be observed, the presence of the bleed has introduced a backward de-compression wave. Also, the backward compression wave has diminished in magnitude and extent.

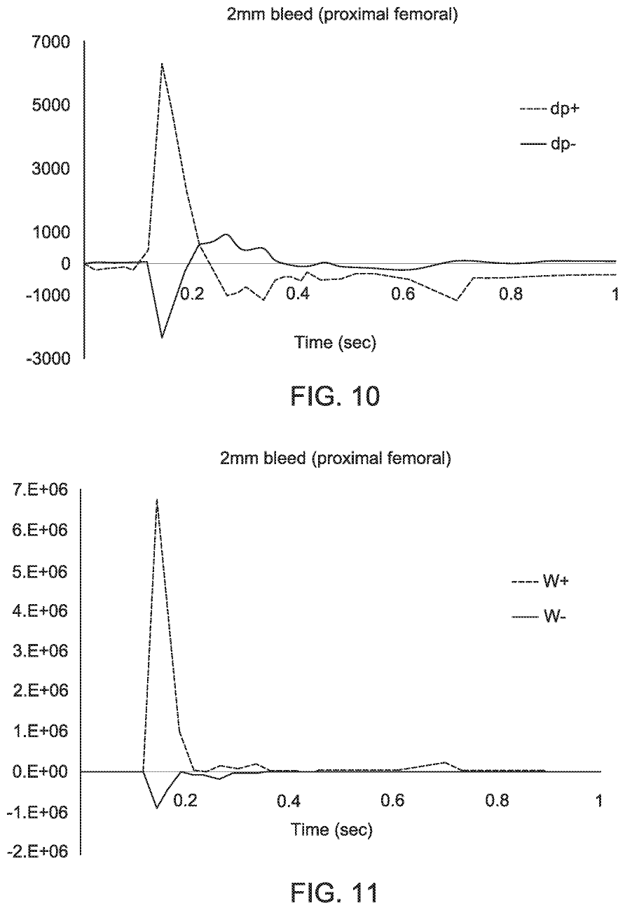

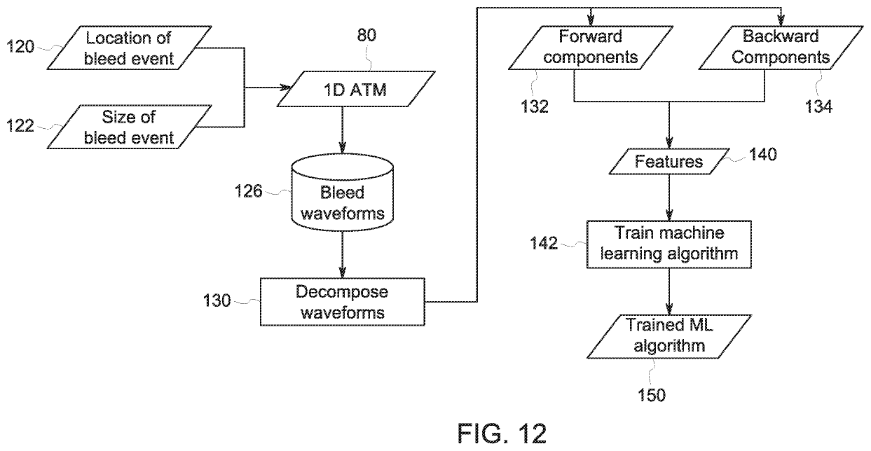

The preceding examples correspond to decompositions of waveforms obtained at a proximal femoral location for a mid-femoral bleed event (i.e., upstream). Waveforms were also obtained and decomposed into forward and backward components for a bleed event close to the proximal femoral location (i.e., a 2 mm bleed in the proximal femoral artery). As shown in FIGS. 10 and 11, in this context the backward de-compression wave is stronger than what is seen for the mid-femoral bleed event and occurs at an earlier time instance. The forward compression wave is also observed to be stronger than what was observed at the proximal location. This increase in observed amplitude and decrease in time until the waves are observed demonstrate that one or both of the amplitude and/or timing of the compression and/or de-compression waves identified in the de-composed waveforms can be used to locate a bleed event.

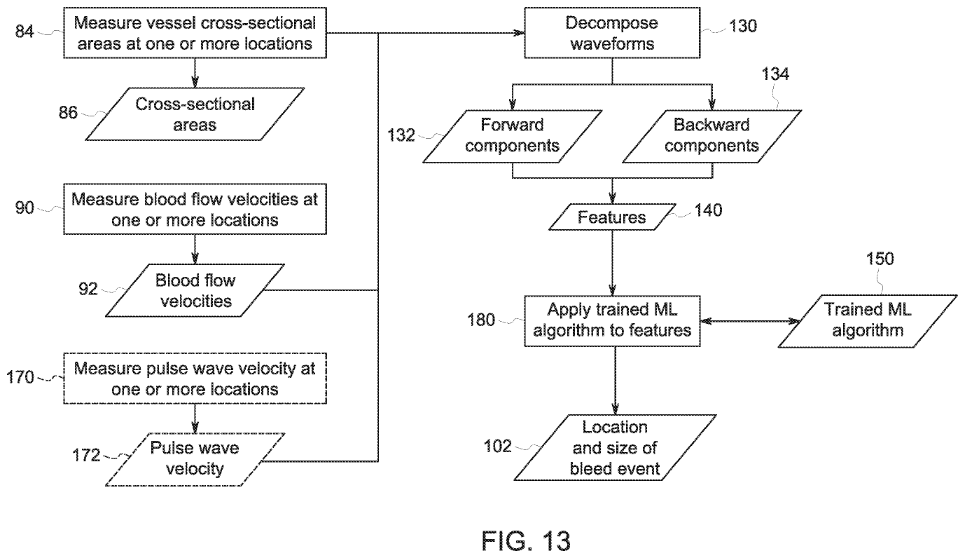

With the preceding in mind, and turning to FIGS. 12 and 13, a method of training a machine learning algorithm is illustrated in FIG. 12 and the use of such a trained algorithm to localize and/or estimate the size of bleed event is illustrated in FIG. 13.

Turning to FIG. 12, a 1D arterial tree model 80 as described herein (or other suitable vasculature model) may be used to generate (i.e., simulate) a set of bleed waveforms 126 (e.g., a library or database of waveforms) corresponding to various specified locations 120 and sizes 122. Alternatively, some or all of the set of bleed waveforms 126 may be generated based on animal or clinical data. The library of bleed waveforms 126 representing different locations and/or sizes of bleed events are decomposed (step 130), such as based on pressure, velocity, artery cross-sectional area, and so forth) into forward components 132 and backward components 134, as discussed herein. From the forward and backward components 132, 134, one or more features 140 may be extracted or identified. Such features 140 may include, but are not limited to, magnitude of the backward de-compression wave, timing of the backward de-compression wave, magnitude of the forward compression wave, and so forth. The features 140 may then be provided as training inputs to a machine learning algorithm 150, such as a neural network, so as to train (step 142) the machine learning algorithm 150. Alternatively, the machine learning algorithm 150 may be trained (step 142) using the forward components 132, backward components 134, or even raw waveforms 126 in addition to or instead of the features 140.

In practice, the trained machine learning algorithm may be used to localize and/or estimate the size or severity of a bleed event based on ultrasound data (e.g., the raw ultrasound data or waveforms, decomposed components of such waveforms, and/or features extracted from the raw or decomposed waveforms) acquired for a patient at one or more landmark locations. By way of example, and turning to FIG. 13, an example of a workflow is provided in which ultrasound data is acquired and process to locate and/or estimate the size of a bleed event. In this example, cross-sectional areas 86 of blood vessels may be measured (step 84) and/or blood flow velocities 92 may be measured (step 90) at one or more landmark location on a patient suspected of having bleeding using ultrasound (e.g., Doppler ultrasound). In an optional implementation, pulse wave velocity 172 may also be measured (step 170) as part of the ultrasound data collection process.

In the depicted example, the acquired ultrasound waveform data is decomposed (step 130) into forward components 132 and backward components 134, as discussed herein. One or more features 140 may be identified in the forward components 132 and/or backward components 134 and the identified features 140 may be provided as an input to the trained machine learning algorithm 150. Based on the provided input, the trained machine learning algorithm 150 outputs (block 102) a location and/or estimated size of a bleed event of the patient. As noted above, though the present example processes feature 140 extracted from decomposed waveforms 132, 134, in practice the trained machine learning algorithm may also or instead process raw versions of the ultrasound data and/or the decomposed waveforms 132, 134 in addition to or instead of the extracted features 140.

As noted above, once the bleed has been detected and accurately localized by either of the techniques described herein, a treatment plan can be formulated and an appropriate therapy applied to limit and/or stop blood loss. Such therapeutic approaches at the point of care may include, but are not limited to, application of pressure or hemostatic pads, as well as deployment of such balloon catheters at the determined location.

Technical effects of the invention include localization of bleeds (e.g., arterial bleed events) using fewer or a minimum number of ultrasound scans. In one implementation, doppler ultrasound is used to measure blood flow velocities in a one-dimensional (1D) arterial tree model to determine the location and size of bleed. In a second implementation, ultrasound measured waveforms for blood flow velocity and vessel cross-sectional area are de-composed into forward and backward components. The features in the de-composed waveforms are then used to locate the bleed using a trained machine learning algorithm.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for localizing a vascular bleed, comprising the acts of:
measuring blood flow velocities at one or more locations on a body of a patient;
fitting the blood flow velocities to vascular tree model; and
based upon the fit of the blood flow velocities to the vascular tree model, determining one or both of a location or a size of a bleed event within the patient.

2. The method of claim 1, wherein measuring blood flow velocities comprises acquiring Doppler ultrasound data.

3. The method of claim 1, wherein the blood flow velocities are represented as one or more blood flow velocity waveforms.

4. The method of claim 1, wherein the vascular tree model comprises a one-dimensional arterial tree model.

5. The method of claim 1, further comprising: constructing the vascular tree model, wherein the vascular tree model is constructed by performing acts comprising:
acquiring vessel cross-sectional areas for the one or more locations; and
constructing the vascular tree model based on the acquired vessel cross-sectional areas.

6. The method of claim 5, wherein acquiring vessel cross sectional areas is performed by one or both of:
measuring vessel cross-sectional areas at the one or more locations using ultrasound; or
deriving the vessel cross-sectional areas based on average or expected human measurements for the vasculature at the one or more locations.

7. The method of claim 1, further comprising: measuring one or both of blood pressure or heart rate of the patient;
wherein one or both of the location or the size of the bleed event within the patient are determined based upon the blood pressure or heart rate in addition to the blood flow velocities.

8. A method for localizing a vascular bleed, comprising the acts of:
generating one or more waveforms using ultrasound imaging, wherein the one or more waveforms describe one or both of vascular vessel cross-sectional area at one or more locations or blood flow velocity at the one or more locations; and
inputting the one or more waveforms or components or features derived from the one or more waveforms to a trained machine learning algorithm, wherein an output of the trained machine learning algorithm is one or both of a location or a size of a bleed event within a patient.

9. The method of claim 8, where pulse wave velocity is also acquired at the one or more locations.

10. The method of claim 8, further comprising:
decomposing the one or more waveforms into forward components and backward components.

11. The method of claim 10, further comprising:
extracting one or more features from the forward components and backward components.

12. The method of claim 8, further comprising: training a machine learning algorithm wherein the machine learning algorithm is trained by performing acts comprising:
accessing a plurality of bleed waveforms from a reference set of bleed waveforms, wherein each bleed waveform corresponds to a bleed event having a bleed size and a bleed location
training the machine learning algorithm using the one or more bleed waveforms or components or features derived from the one or more bleed waveforms and the bleed size and bleed location corresponding to each bleed event to generate a trained machine learning algorithm

13. The method of claim 12, further comprising:
decomposing the one or more bleed waveforms into forward components and backward components of the respective bleed waveforms.

14. The method of claim 13, further comprising:
extracting one or more features from the forward components and backward components of the respective bleed waveforms.

15. The method of claim 12, wherein the reference set of bleed algorithms is generated using one or both of clinical or animal bleed data or simulations performed using a vascular tree model.

16. A system for localizing bleed events, comprising:
an ultrasound scanner configured to generate ultrasound data at one or more locations of a body of a patient;
a memory component configured to store one or more processor-executable routines; and
a processing component configured to receive or access the ultrasound data and to execute the one or more processor-executable routines, wherein the one or more routines, when executed by the processing component, cause the processing component to perform acts comprising:
measuring at least blood flow velocities at one or more locations on a body of a patient;
processing at least the blood flow velocities or one or more components or features derived from at least the blood flow velocities to determine one or both of a location or a size of a bleed event within the patient.

17. The system of claim 16, wherein processing at least the blood flow velocities or one or more components or features derived from at least the blood flow velocities comprises: fitting the blood flow velocities to a vascular tree model and, based upon the fit of the blood flow velocities to the vascular tree model, determining one or both of the location or the size of the bleed event.

18. The system of claim 16, wherein processing at least the blood flow velocities or one or more components or features derived from at least the blood flow velocities comprises: inputting one or more waveforms corresponding to the blood flow velocities or components or features derived from the one or more waveforms to a trained machine learning algorithm, wherein an output of the trained machine learning algorithm is one or both of the location or the size of the bleed event.

19. The system of claim 18, wherein processing at least the blood flow velocities or one or more components or features derived from at least the blood flow velocities comprises also processing one or more additional waveforms corresponding to the vascular vessel cross-sectional area or components or features derived from the one or more additional waveforms using the trained machine learning algorithm.

20. The system of claim 16, wherein the one or more components comprise forward components and backward components of a waveform and the features comprise one or more features extracted from one or both of the forward components and the backward components.
